(19) United States
(12) Patent Application Publication
(10) Pub. No.: US 2024/0074646 A1
(43) Pub. Date: Mar. 7, 2024

(54) IMAGING UNIT AND ENDOSCOPE

(71) Applicant: OLYMPUS MEDICAL SYSTEMS CORP., Tokyo (JP)

(72) Inventor: Yuki Ishii, Tokyo (JP)

(73) Assignee: OLYMPUS MEDICAL SYSTEMS CORP., Tokyo (JP)

(21) Appl. No.: 18/242,732

(22) Filed: Sep. 6, 2023

Related U.S. Application Data
(60) Provisional application No. 63/404,216, filed on Sep. 7, 2022.

(51) Int. Cl. A61B 1/05; A61B 1/06

(52) U.S. Cl. CPC A61B 1/051; A61B 1/0676

(57) ABSTRACT

An imaging unit includes: an illumination unit that includes an illumination optical system; a first holding frame configured to hold the illumination unit; an imaging-unit main body that includes an imager, and an observation optical system configured to form the subject image on the imager; and a second holding frame configured to hold the imaging-unit main body. A first abutting portion configured to abut on the second holding frame in a radial direction of the first holding frame is arranged on an outer surface of the first holding frame.

### CROSS-REFERENCE TO RELATED APPLICATION

This application is based on and claims priority under 35 U.S.C. § 119 to U.S. Provisional Application No. 63/404,216, filed Sep. 7, 2022, the entire contents of which are incorporated herein by reference.

### BACKGROUND

### 1. Technical Field

The present disclosure relates to an imaging unit and an endoscope.

### 2. Related Art

In the related art, an endoscope in which an imaging unit is arranged at a distal end of an insertion portion inserted into an inside of a subject has been known (for example, refer to JP-A-2012-152390).

An imaging unit described in JP-A-2012-152390 has a structure in which an illumination unit and an imaging-unit main body are held adjacent to each other by a distal end cover and a distal-end rigid portion. The illumination unit includes a light emitting device and an illumination optical system that emits light from the light emitting device to a subject. The imaging-unit main body includes an imager that captures a subject image, and an observation optical system that forms an image of the subject image on the imager.

### SUMMARY

In some embodiments, an imaging unit includes: an illumination unit that includes an illumination optical system; a first holding frame configured to hold the illumination unit; an imaging-unit main body that includes an imager, and an observation optical system configured to form the subject image on the imager; and a second holding frame configured to hold the imaging-unit main body. A first abutting portion configured to abut on the second holding frame in a radial direction of the first holding frame is arranged on an cuter surface of the first holding frame, when a range holding the illumination unit in a direction along an optical axis of the illumination optical system in the first holding frame is a first axial-direction range, and a range in the direction along the optical axis different from the first axial-direction range is a second axial-direction range, the first abutting portion is arranged in the second axial-direction range, and a cavity is formed by the first abutting portion in the first axial-direction range between the first holding frame and the second holding frame.

In some embodiments, an endoscope includes: an insertion portion configured to be inserted into an inside of a subject body; and an imaging unit that is arranged at a distal end of the insertion portion. The imaging unit includes an illumination unit that includes an illumination optical system configured to irradiate light to a subject; a first holding frame configured to hold the illumination unit; an imaging-unit main body that includes an imager configured to capture a subject image, and an observation optical system configured to form the subject image on the imager; and a second holding frame configured to hold the imaging-unit main body, on an outer surface of the first holding frame, a first abutting portion configured to abut on the second holding frame in a radial direction of the first holding frame is arranged, when a range holding the illumination unit in a direction along an optical axis of the illumination optical system in the first holding frame is a first axial-direction range, and a range in the direction along the optical axis different from the first axial-direction range is a second axial-direction range, the first abutting portion is arranged in the second axial-direction range, and a cavity is formed by the first abutting portion in the first axial-direction range between the first holding frame and the second holding frame.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating a configuration of an endoscope system according to a first embodiment;

FIG. 2 is a diagram illustrating a configuration of an imaging unit;

FIG. 3 is a diagram illustrating a shape of a first abutting portion;

FIG. 4 is a diagram illustrating a shape of a second abutting portion;

FIG. 5 is a diagram illustrating a configuration of an imaging unit according to a second embodiment;

FIG. 6 is a diagram illustrating a configuration of an imaging unit according to a third embodiment;

FIG. 7 is a diagram illustrating a configuration of a proximal end side illumination lens;

FIG. 8 is a diagram illustrating a configuration of an imaging unit according to a fourth embodiment;

FIG. 9 is a diagram illustrating a configuration of an imaging unit according to a fifth embodiment;

FIG. 10 is a diagram explaining a first modification of the first to the fifth embodiments;

FIG. 11 is a diagram explaining the first modification of the first to the fifth embodiments;

FIG. 12 is a diagram explaining a second modification of the first to the fifth embodiments;

FIG. 13 is a diagram explaining the second modification of the first to the fifth embodiments;

FIG. 14 is a diagram explaining a third modification of the first to the fifth embodiments; and

FIG. 15 is a diagram explaining a fourth modification of the first to the fifth embodiments.

### DETAILED DESCRIPTION

Hereinafter, forms (hereinafter, embodiments) to implement the disclosure will be explained with reference to the drawings. The embodiments explained below are not intended to limit the disclosure. Furthermore, identical signs are assigned to identical parts throughout the descriptions of the drawings.

### First Embodiment

Configuration of Endoscope System

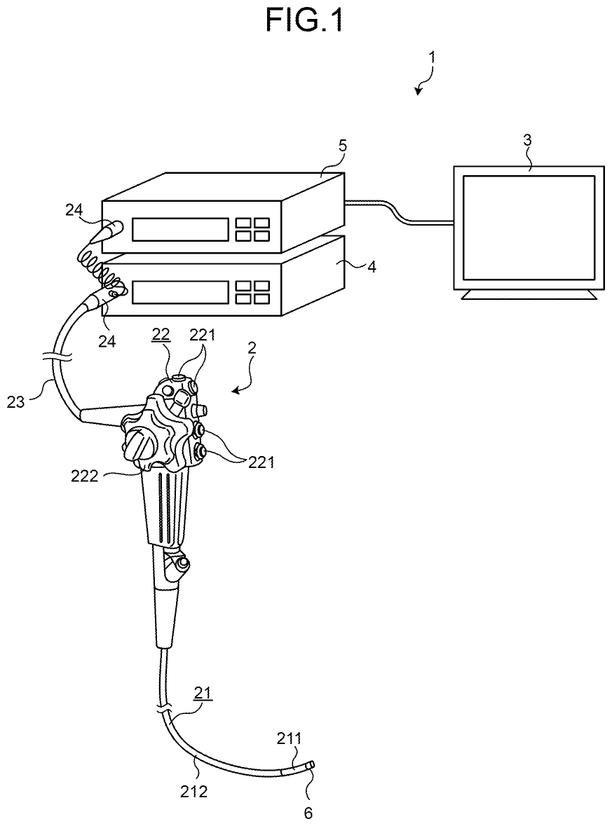

FIG. 1 is a diagram illustrating a configuration of an endoscope system 1 according to a first embodiment.

An endoscope system 1 is used in, for example, the medical field, and is a system to observe an inside of a subject (inside living body). This endoscope system 1 includes, as illustrated in FIG. 1, an endoscope 2, a display device 3, a light source device 4, and a control device 5.

A portion of the endoscope 2 is inserted into an inside of a living body, images the inside of the living body, and outputs an image signal generated by the imaging. This endoscope 2 includes, as illustrated in FIG. 1, an insertion portion 21, an operating unit 22, a universal cord 23, and a connector portion 24.

The insertion portion 21 has flexibility in at least a part thereof, and is a portion to be inserted in to an inside of a living body. This insertion portion 21 includes, as illustrated in FIG. 1, an imaging unit 6, a bendable portion 211, and a flexible tube 212.

To explain a configuration of the insertion portion 21, “distal end side” described hereinafter is a distal end in an insertion direction of the insertion portion 21, and signifies a subject side. Furthermore, “proximal end side” described hereinafter, signifies aside apart from the distal end side, in other words, a side apart from the subject.

The imaging unit 6 is arranged at a distal end of the insertion portion 21. The imaging unit 6 images a subject image, and outputs an image signal generated from the imaging.

A detailed configuration of the imaging unit 6 will be explained in “Configuration of imaging Unit” described later.

The bendable portion 211 is connected to a proximal end side of the imaging unit 6. Although specific illustration thereof is omitted, this bendable portion 211 has a structure in which plural bend pieces are connected, to be bendable.

The flexible tube 212 is a long portion having flexibility, and is connected to a proximal end side of the bendable portion 211.

The operating unit 22 is connected to a proximal end of the insertion portion 21. The operating unit 22 accepts various kinds of operations with respect to the endoscope 2. In this operating unit 22, as illustrated in FIG. 1, plural operating portions 221, and a bending knob 222 are provided.

The operating portions 221 are constituted of buttons that accept various kinds of operations, and the like.

The bending knob 222 is configured to be rotatable according to a user operation. The bending knob 222 rotates, to operate a bending mechanism (not illustrated), such as a metal or resin wire, arranged inside the insertion portion 21. Thus, the bendable portion 211 bends.

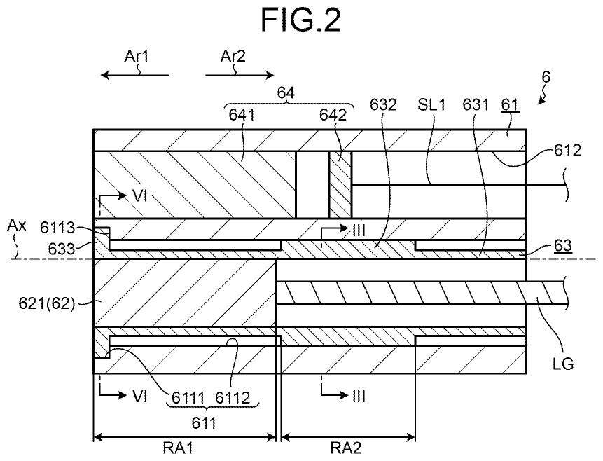

The universal cord 23 is a cord extending from the operating unit 22 in a direction different from an extending direction of the insertion portion 21. In this universal cord 23, a light guide LG (refer to FIG. 2) that guides illumination light supplied from the light source device 4 to the imaging unit 6, a signal line SL1 (refer to FIG. 2) that transmits an image signal output from the imaging unit 6, and the like are arranged.

The connector portion 24 is arranged at an end portion of the universal cord 23, and is detachably connected to the light source device 4 and the control device 5.

The display device 3 is a liquid crystal display (LCD), an electroluminescence (EL) display, or the like, and displays a predetermined image under control of the control device 5.

The light source device 4 supplies illumination light. The illumination light from the light source device 4 is irradiated to the inside of a living body from the distal end of the insertion portion 21 after passing through the connector portion 24, the universal cord 23, the operating unit 22, the light guide LG drawn to the insertion portion 21, and the imaging unit 6.

The control device 5 includes a processor, such as a central processing unit (CPU) and a microprocessor unit (MPU), and comprehensively controls operations of the display device 3 and the light source device 4. The processor may be constituted of an integrated circuit, such as an application specific integrated circuit (ASIC) and a field programmable gate array (FPGA), not limited to the CPU or MPU.

In the first embodiment, the light source device 4 and the control device 5 are configured separately, but it is not limited thereto, and may be provided integrally in a single casing.

### Configuration of Imaging Unit

Next, a configuration of the imaging unit 6 described above will be explained.

FIG. 2 is a diagram illustrating a configuration of the imaging unit 6. Specifically, FIG. 2 is a cross-section of the imaging unit 6 cut along a plane including a center axis Ax along a longitudinal direction in the insertion portion 21. Moreover, in FIG. 2, a reference symbol “Ar1” signifies a distal end side. A reference sign “Ar2” signifies a proximal end side.

The imaging unit 6 includes, as illustrated in FIG. a distal-end rigid portion 61, an illumination unit 62, an illumination lens barrel 63, and an imaging-unit main body 64.

The distal-end rigid portion 61 corresponds to a second holding frame. This distal-end rigid portion 61 is a rigid portion constituted of, for example, a resin material, and has a substantially cylindrical shape extending along the center axis Ax.

In this distal-end rigid portion 61, as illustrated in FIG. 2, a first and a second through holes 611, 612 that respectively pierce through from an end surface of the proximal end to an end surface of the distal end along the center axis Ax are formed.

The first through hole 611 is a through hole in which the illumination unit 62 and the illumination lens barrel 63 are arranged as illustrated in FIG. 2, and includes a large diameter hole 6111 and a small diameter hole 6112.

The large diameter hole 6111 is arranged on the distal end side Ar1 of the small diameter hole 6112 as illustrated in FIG. 2, and has a circular cross-sectional shape.

The small diameter hole 6112 is arranged coaxially with the large diameter hole 6111 as illustrated in FIG. 2, and has a cross-sectional circular shape, an inner diameter of which is smaller than the large diameter hole 6111.

The first through hole 611 has a stepped surface 6113 connecting the large diameter hole 6111 and the small diameter hole 6112 as illustrated in FIG. 2. The stepped surface 6113 is constituted of a flat surface perpendicular to the center axis Ax.

The second through hole 612 is a through hole in which the imaging-unit main body 64 is arranged as illustrated in FIG. 2, and has a substantially circular shape on its cross section.

In the first embodiment, the illumination unit 62 is constituted only of an illumination optical system 621. This illumination optical system 621 is constituted of one or more lenses, and is arranged such that optical axes are parallel with the center axis Ax. The illumination optical system 621 opposes to an emitting end of the light guide LG drawn in the insertion portion 21, and irradiates light transmitted by the light guide LG to the inside of the subject from the distal end of the insertion portion 21.

The illumination lens barrel 63 holds the illumination unit 62. This illumination lens barrel 63 is constituted of, for example, a resin material, and is a portion in which a lens-barrel main body 631 and the first and second abutting portions 632 and 633 are integrally configured as illustrated in FIG. 2. For the illumination lens barrel 63, a configuration in which the lens-barrel main body 631 and the first and second abutting portions 632 and 633 are configured separately, and are assembled with each other and then fixed to each other by adhesive or the like may be adopted.

The lens-barrel main body 631 corresponds to a first holding frame. This lens-barrel main body 631 has a cylindrical shape having an outer diameter smaller than an inner diameter of the small diameter hole 6112, holds the illumination unit 62 thereinside on a distal end side and is inserted into the first through hole 611. The lens-barrel main body 631 is arranged in the first through hole 611 in such a position that its center axis is parallel to the center axis Ax.

FIG. 3 is a diagram illustrating a shape of the first abutting portion 632. Specifically, FIG. 3 is a cross-section taken along a line indicated in FIG. 2. In FIG. 3, only the illumination lens barrel 63 is illustrated for convenience of explanation.

The first abutting portion 632 is arranged on an outer peripheral surface of the lens-barrel main body 631 as illustrated in FIG. 2 and FIG. 3, and has a ring shape extending throughout an entire circumferential direction centered around the center axis of the lens-barrel main body 631. An outer diameter of this first abutting portion 632 is substantially the same as an inner diameter of the first through hole 611. The first abutting portion 632 abuts on an inner peripheral surface of the first through hole 611 (small diameter hole 6112) when the lens-barrel main body 631 is inserted into the first through hole 611. Thus, a cavity is formed by the first abutting portion 632 in a first axial-direction range RA between the outer peripheral surface of the lens-barrel main body 631 and the inner peripheral surface of the first through hole 6111. Moreover, the first abutting portion 632 determines a position of the lens-barrel main body 631 in a radial direction by abutting on the inner peripheral surface of the first through hole 611.

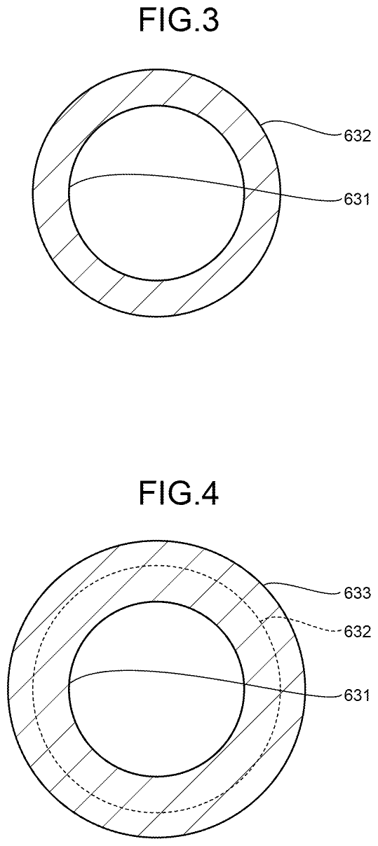

FIG. 4 is a diagram illustrating a shape of the second abutting portion 633. Specifically, FIG. 4 is a cross-section taken along a line IV-IV indicated in FIG. 2. In FIG. 4, only the illumination lens barrel 63 is illustrated for convenience of explanation.

The second abutting portion 633 is arranged on the outer peripheral surface of the lens-barrel main body 631, and has a ring shape extending throughout an entire circumferential direction centered around the center axis of the lens-barrel main body 631 as illustrated in FIG. 2 and FIG. 4. An outer diameter of this second abutting portion 633 is larger than the inner diameter of the first through hole 611. An end surface of this second abutting portion 633 on the proximal end side Ar2 is constituted of a flat surface perpendicular to the center axis of the lens-barrel main body 631. The second abutting portion 633 abuts on the stepped surface 6113 when the lens-barrel main body 631 is inserted into the first through hole 611 from the distal end side Ar1 of the distal-end rigid portion 61. Thus, the second abutting portion 633 determines a position of the lens-barrel main body 631 in a direction along the center axis Ax.

The imaging-unit main body 64 includes, as illustrated in FIG. 2, an observation optical system 641 and an imager 642.

The observation optical system 641 is constituted of one or plural lenses, and is arranged inside the second through hole 612 on the distal end side Ar1 such that optical axes are parallel with the center axis Ax. The observation optical system 641 captures returning light that has been irradiated to the inside of a living body from the illumination unit 62, and returned from the inside of the living body (subject image), and forms an image on an imaging surface of the imager 642.

The imager 642 is arranged inside the second through hole 612 on the proximal end side Ar2 relative to the observation optical system 641 as illustrated in FIG. 2. This imager 642 is an imager, such as a charge coupled device (CCD) or a complementary metal oxide semiconductor (CMOS), and images a subject image formed by the observation optical system 641, and outputs an image signal generated by the imaging. The control device 5 acquires the image signal output from the imager 642 through the signal line SL1. Furthermore, the control device 5 generates a video signal for display by subjecting the image signal to various kinds of image processing, and outputs it to the display device 3. Thus, the display device 3 displays an image based on the image signal generated by the imager 642.

### Arrangement Position of First Abutting Portion

Next, an arrangement position of the first abutting portion 632 will be explained referring to FIG. 2.

Hereinafter, a range in which the illumination unit 62 is held in a direction along the center axis Ax in the lens-barrel main body 631 is denoted as a first axial-direction range RA1 (FIG. 2). Moreover, a range in a direction along the center axis Ax in which the first abutting portion 632 is arranged in the lens-barrel main body 631 is denoted as a second axial-direction range RA2 (FIG. 2).

The second axial-direction range RA2 is a range different from the first axial-direction range RA1 as illustrated in FIG. 2. In the first embodiment, the second axial-direction range RA2 is a range on the proximal end side Ar2 relative to the first axial-direction range RA1. Moreover, although the second axial-direction range RA2 does not overlap the first axial-direction range RA1, it overlaps the proximal end side Ar2 at the arrangement position of the observation optical system 641, and the arrangement position of the imager 642, respectively.

A length dimension (the second axial-direction range RA2) in a direction along the center axis Ax of the first abutting portion 632 may be shorter than the first axial-direction range RA1, or may be equal to or longer than the first axial-direction range RA1. Furthermore, a length dimension in a direction along the center axis Ax of the second abutting portion 633 is shorter than ½ of the first axial-direction range RA1.

According to the first embodiment, following effects are produced.

In the imaging unit 6 according to the first embodiment, the first abutting portion 632 abutting on the inner surface of the first through hole 611 (the small diameter hole 6112) is arranged in the second axial-direction range RA2 on the outer surface of the lens-barrel main body 631. Between the outer peripheral surface of the lens-barrel main body 631 and the inner peripheral surface of the first through hole 611, a cavity is formed by the first abutting portion 632 in the first axial-direction range RA1. Particularly, the second axial-direction range RA2 is a range on the proximal end side Ar2 relative to the first axial-direction range RA1. That is, by the cavity, a linear heat transfer path from the illumination unit 62 through the illumination lens barrel 63 to the imaging-unit main body 64 is narrowed.

Therefore, according to the imaging unit 6 according to the first embodiment, it is possible to suppress transmission of heat generated in the illumination optical system 621 by light emitted from the light guide LG to the imaging-unit main body 64.

Moreover, in the imaging unit 6 according to the first embodiment, the first abutting portion 632 abuts on the inner peripheral surface of the first through hole 611, and thereby determines a position of the lens-barrel main body 631 in a radial direction.

Therefore, it is possible to add a function as a positioning portion to the first abutting portion 632, and it is not necessary to provide a positioning portion separately, and simplification of the shape of the illumination lens barrel 63 is possible.

### Second Embodiment

Next, a second embodiment will be explained.

In the following explanation, identical signs are assigned to components identical to those of the first embodiment described above, and detailed explanation thereof is omitted or simplified.

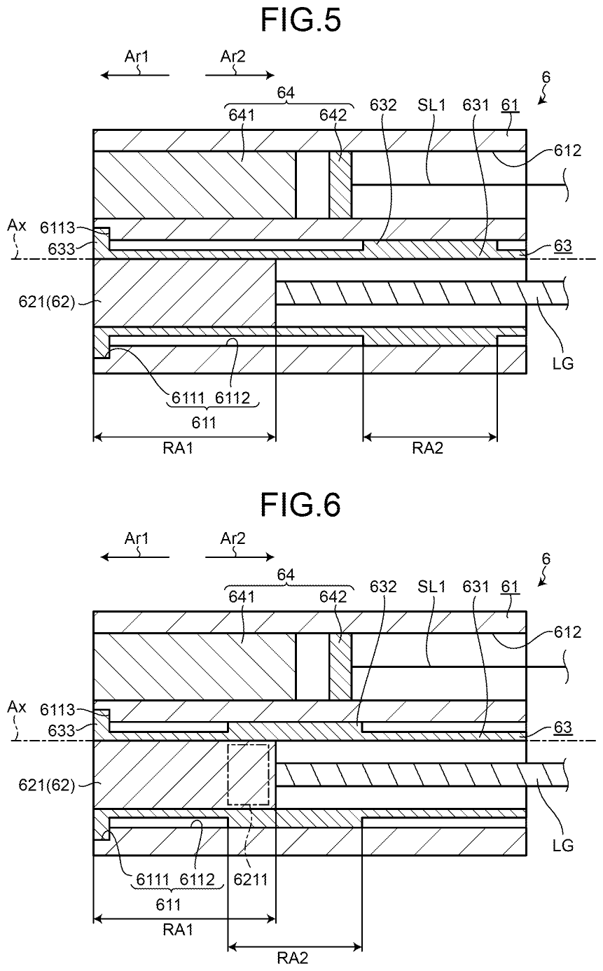

FIG. 5 is a diagram illustrating a configuration of the imaging unit 6 according to the second embodiment. Specifically, FIG. 5 is a diagram corresponding to FIG. 2.

In the second embodiment, as illustrated in FIG. 5, an arrangement position (the second axial-direction range RA2) of the first abutting portion 632 is changed from the first embodiment described above.

The second axial-direction range RA2 according to the second embodiment is a range on the proximal end side Ar2 relative to the arrangement position of the imager 642 that is arranged on the proximal end side Ar2 relative to the first axial-direction range RA1 as illustrated in FIG. 5.

A length dimension (the second axial-direction range RA2) in a direction along the center axis Ax of the first abutting portion 632 according to the second embodiment may be shorter than the first axial-direction range RA1 similarly to the first embodiment described above, or may be equal to or longer than the first axial-direction range RA1.

According to the second embodiment explained above, a following effect is produced other than effects similar to those of the first embodiment described above.

In the imaging unit 6 according to the second embodiment, the second axial-direction range RA2 is a range on the proximal end side Ar2 relative to the arrangement position of the imager 642.

Therefore, between the outer peripheral surface of the lens-barrel main body 631 and the inner peripheral surface of the first through hole 611, a range of the cavity formed by the first abutting portion 632 in the first axial-direction range RA1 can be further expanded. That is, b the cavity, the linear heat transfer path from the illumination unit 62 through the illumination lens barrel 53 to the imaging-unit main body 64 can be further narrowed. Therefore, it is possible to further suppress heat transfer from the illumination unit 62 to the imaging-unit main body 64.

### Third Embodiment

Next, a third embodiment will be explained.

In the following explanation, identical signs are assigned to components similar to those of the first embodiment described above, and detailed explanation is omitted or simplified.

FIG. 6 is a diagram illustrating a configuration of the imaging unit 6 according to the third embodiment. Specifically, FIG. 6 is a diagram corresponding to FIG. 2.

In the third embodiment, as illustrated in FIG. 6, the arrangement position of the first abutting portion 632 (the second axial-direction range RA2) is changed from the first embodiment described above as illustrated in FIG. 6.

The illumination optical system 621 according to the third embodiment is constituted of plural lenses. Out of the plural lenses, a proximal-end-side illumination lens 6211 that is positioned on the most proximal end side has a following configuration.

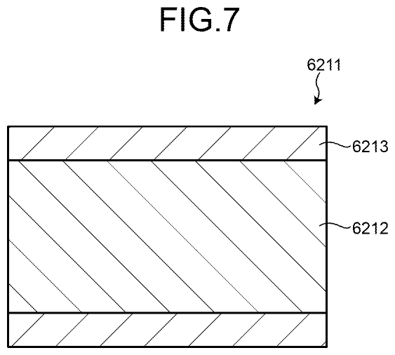

FIG. 7 is a diagram illustrating a configuration of the proximal-end-side illumination lens 6211.

Specifically, FIG. 7 is a cross-section of the proximal-end-side illumination lens 6211 cut along a plane including an optical axis of the proximal-end-side illumination lens 6211 (plane including the center axis Ax).

The proximal-end-side illumination lens 6211 is a lens constituted of two layers of a core portion 6212 and a cladding portion 6213 arranged on an outer circumference of the core portion 6212 as illustrated in FIG. 7. Light emitted from an emitting end of the light guide LG is irradiated, after passing through only the core portion 6212 in the proximal-end-side illumination lens 6211, to the inside of a living body from the distal end of the insertion portion 21 through the other lenses in the illumination optical system 621.

The second axial-direction range RA2 according to the third embodiment is a range overlapping at least a part of the arrangement position of the proximal-end-side illumination lens 6211. In other words, when dividing the first axial-direction range RA1 evenly into two ranges of a range on the distal end side and a range on the proximal end side, the second axial-direction range RA2 overlaps at least a part of the range on the proximal end side.

A length dimension (the second axial-direction range RA2) in a direction along the center axis Ax of the first abutting portion 632 according to the third embodiment may be shorter than the first axial-direction range RA1 similarly to the first embodiment described above, or may be equal to or larger than the first axial-direction range RA1.

According to the third embodiment explained above, a following effect is produced.

In the imaging unit 6 according to the third embodiment, the second axial-direction range RA2 is a range overlapping at least a part of the arrangement position of the proximal-end-side illumination lens 6211. The proximal-end-side illumination lens 6211 is a lens constituted of two layers of the core portion 6212 and the cladding portion 6213, and a temperature increase of the cladding portion 6213 by light emitted from the light guide LG is small.

Therefore, also when the second axial-direction range RA2 is set in a range overlapping at least a part of the arrangement position of the proximal-end-side illumination lens 6211, an effect similar to that of the first embodiment is produced.

### Fourth Embodiment

Next, a fourth embodiment will be explained.

In the following explanation, identical signs are assigned to components similar to those of the first embodiment, and detailed explanation is omitted or simplified.

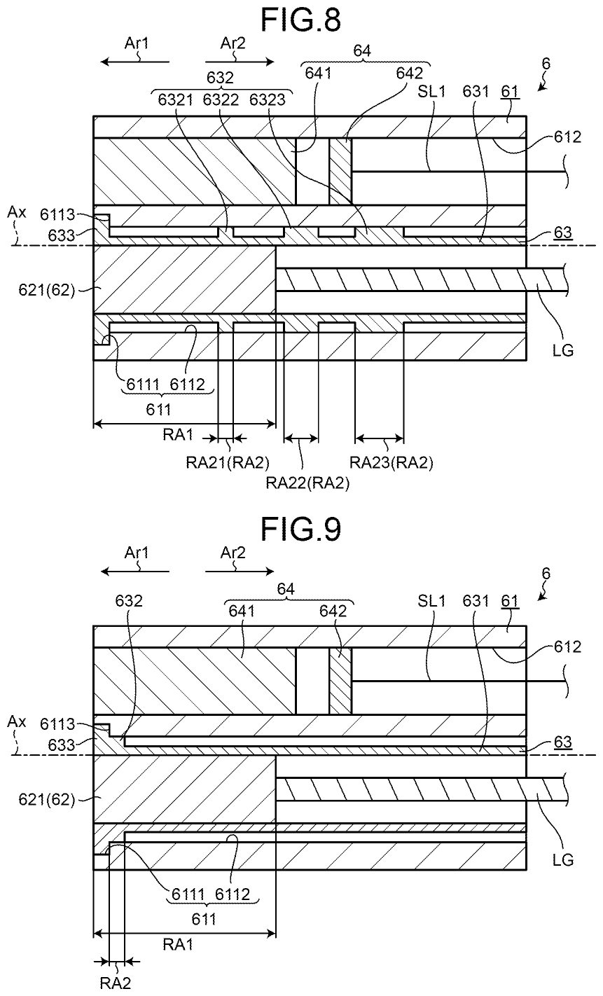

FIG. 8 is a diagram illustrating a configuration of the imaging unit 6 according to the fourth embodiment. Specifically, FIG. 8 is a diagram corresponding to FIG. 2.

In the fourth embodiment, as illustrated in FIG. 8, the number of the first abutting portion 632 is changed from the first embodiment.

The first abutting portion 632 according to the fourth embodiment is arranged in plurality (three in the fourth embodiment) separated from one another along the center axis Ax as illustrated in FIG. 8.

In the following, out of the three first abutting portions 632, the first abutting portion 632 positioned on the most distal end side Ar1 is denoted as distal-end-side abutting portion 6321, the first abutting portion 632 positioned second from the distal end side Ar1 is denoted as intermediate abutting portion 6322, and the first abutting portion 632 positioned on the most proximal end side Ar2 is denoted as proximal-end-side abutting portion 6323 (FIG. 8). Moreover, out of the second axial-direction range RA2, a range in which the distal-end-side abutting portion 6321 is arranged is denoted as distal-end-side range RA21, a range in which the intermediate abutting portion 6322 is arranged is denoted as intermediate range RA22, and a range in which the proximal-end-side abutting portion 6323 is arranged is denoted as proximal-end-side range RA23 (FIG. 8).

The distal-end-side range RA21 is a range overlapping a part of the first axial-direction range RA1 as illustrated in FIG. 8. More specifically, the distal-end-side range RA21 overlaps, when dividing the first axial-direction range RA1 into two equally between a range on a distal end side and a range on a proximal end side, at least a part of the range on the proximal end side.

A length dimension (the distal-end-side range RA21) in a direction along the center axis Ax of the distal-end-side abutting portion 6321 is equal to or smaller than ½ of the first axial-direction range RA1.

The intermediate range RA22 is a range on the proximal end side Ar2 relative to the first axial-direction range RA1 and on the distal end side Ar1 relative to the arrangement position of the imager 642 as illustrated in FIG. 8.

The proximal-end-side range RA23 is a range on the proximal end side Ar2 relative to the arrangement position of the imager 642 as illustrated in FIG. 8.

A length dimension (the intermediate range RA22 and the proximal-end-side range RA23) in a direction along the center axis Ax of the intermediate abutting portion 6322 and the proximal-end-side abutting portion 6323 may be shorter than the first axial-direction range RA1, or may be equal to or longer than the first axial-direction range RA1.

According to the fourth embodiment explained above, a following effect is produced.

In the imaging unit 6 according to the fourth embodiment, the second axial-direction range RA2 is constituted of three ranges, the distal-end-side range RA21 that overlaps a part of the first axial-direction range RA1, the intermediate range RA22 and the proximal-end-side range RA23 that are ranges on the proximal end side Ar2 relative to the first axial-direction range RA1. The distal-end-side range RA21 is equal to or smaller than ½ of the first axial-direction range RA1. That is, the distal-end-side abutting portion 6321 has such a length dimension that does not contribute significantly to heat transfer from the illumination unit 62 to the imaging-unit main body 64.

Therefore, also when a part of the second axial-direction range RA2 (the distal-end-side range RA21) is set in a range overlapping a part of the first axial-direction range RA1, an effect similar to that of the first embodiment is produced.

### Fifth Embodiment

Next, a fifth embodiment will be explained.

In the following explanation, identical signs are assigned to components similar to those of the first embodiment, and detailed explanation is omitted or simplified.

FIG. 9 is a diagram illustrating a configuration of the imaging unit 6 according to the fifth embodiment. Specifically, FIG. 9 is a diagram corresponding to FIG. 2.

In the fifth embodiment, as illustrated in FIG. 9, the arrangement position (the second axial-direction range RA2) of the first abutting portion 632 is changed from the first embodiment described above.

The first abutting portion 632 according to the fifth embodiment is integrally formed with the second abutting portion 633 as illustrated in FIG. 9. The second axial-direction range RA2 according to the fifth embodiment overlaps, when dividing the first axial-direction range RA1 evenly into two ranges of a range on the distal end side and a range on the proximal end side, at least a part of the range on the distal end side as illustrated in FIG. 9.

A length dimension (the second axial-direction range RA2) in a direction along the center axis Ax of the first abutting portion 632 according to the third embodiment is equal to or smaller than ½ of the first axial-direction range RA1.

According to the fifth embodiment explained above, a following effect is produced.

In the imaging unit 6 according to the fifth embodiment, the second axial-direction range RA2 is a range overlapping a part of the first axial-direction range RA1. The length dimension in a direction along the center axis Ax of the first abutting portion 632 is equal to or smaller than ½ of the first axial-direction range RA1. That is, the first abutting portion 632 has a length dimension that does not contribute significantly to heat transfer from the illumination unit 62 to the imaging-unit main body 64.

Therefore, also when the second axial-direction range RA2 is set in a range overlapping a part of the first axial-direction range RA1, an effect similar to that of the first embodiment is produced.

Particularly, in the imaging unit 6 according to the fifth embodiment, the first and second abutting portions 632 and 633 are formed integrally. Accordingly, the second axial-direction range RA2 can be set at a position apart from the arrangement position of the imager 642. Therefore, also when the second axial-direction range RA2 is set in a range overlapping a part of the first axial-direction range RA1, a temperature increase of the imager 642 by heat from the illumination unit 62 can be suppressed.

### Other Embodiments

The embodiments to implement the disclosure have so far been explained, but the disclosure is not to be limited to the first to the fifth embodiments described above.

Although, the endoscope 2 is used in the medical field in the first to the fifth embodiments described above, it is not limited thereto, and may be used in an industrial field.

### First Modification

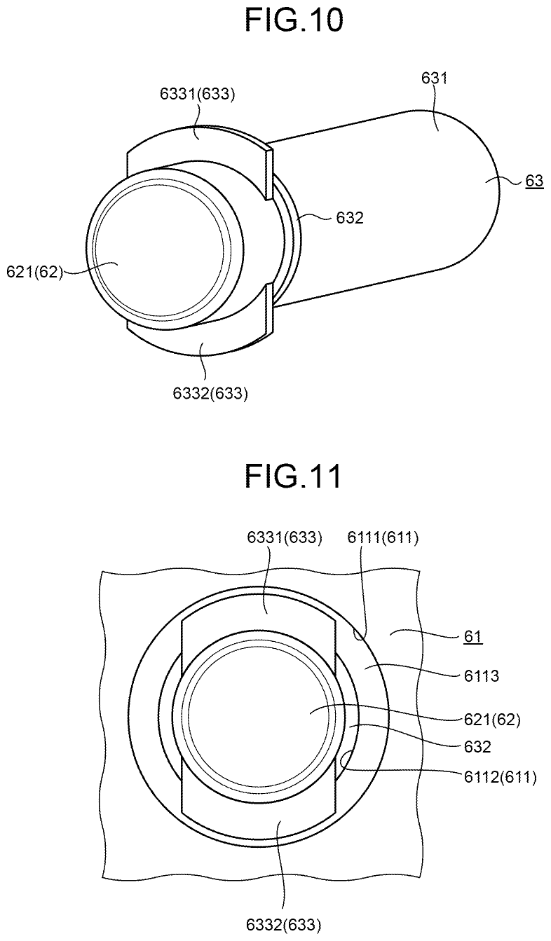

FIG. 10 and FIG. 11 are diagrams explaining a first modification of the first to the fifth embodiments. Specifically, FIG. 10 is a perspective view of the illumination lens barrel 63 and the illumination unit 62 according to the first modification viewed from the distal end side Ar1. FIG. 11 is a diagram of the illumination lens barrel 63 arranged at the distal-end rigid portion 61 according to the first modification viewed from the distal end side Ar1 along the center axis Ax.

In the first to the fifth embodiments described above, the second abutting portion 633 has a ring shape extending throughout an entire circumferential direction centered around the center axis of the lens-barrel main body 631, but not limited thereto, a shape of the first modification illustrated in FIG. 10 and FIG. 11 may be adopted.

The second abutting portion 633 according to the first modification is arranged in two pieces. In the following, the two abutting portions 633 are denoted as the second abutting portions 6331, 6332 (FIG. 10, FIG. 11). These two second abutting portions 6331, 6332 respectively have an arc shape extending along a circumferential direction centered around the center axis of the lens-barrel main body 631, and are respectively positioned rotationally symmetrically by 180° about the center axis.

While FIG. 10 and FIG. 11 illustrate a case in which the second abutting portion 633 according to the first modification is applied to the illumination lens barrel 63 explained in the fifth embodiment, it may be applied to the illumination lens barrel 63 explained in the first to the fourth embodiments described above.

Also when the second abutting portion 633 according to the first modification explained above is adopted, an effect similar to those of the first to the fifth embodiments is produced.

### Second Modification

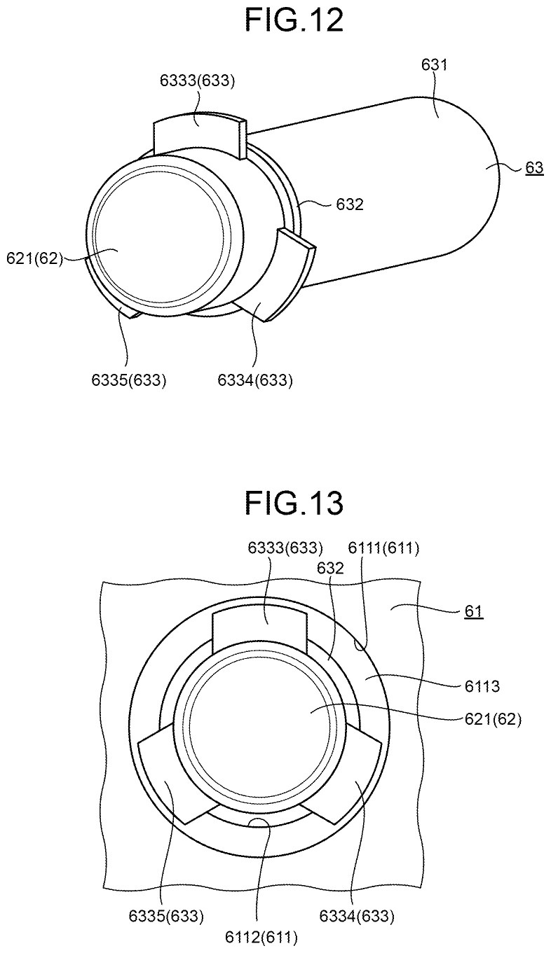

FIG. 12 and FIG. 13 are diagrams explaining a second modification of the first to the fifth embodiments. Specifically, FIG. 12 is a perspective view of the illumination lens barrel 63 and the illumination unit 62 according to the second modification viewed from the distal end side Ar1. FIG. 13 is a diagram of the illumination lens barrel 63 arranged at the distal-end rigid portion 61 according to the second modification viewed from the distal end side Ar1 along the center axis Ax.

Although the second abutting portion 633 has a ring shape extending throughout the entire circumferential direction centered around the center axis of the lens-barrel main body 631 in the first to the fifth embodiments, it is not limited thereto, and a shape of the second modification illustrated in FIG. 12 and FIG. 13 may be adopted.

The second abutting portions 633 according to the second modification is arranged in three pieces. In the following, the three second abutting portions 633 are denoted as second abutting portions 6333 to 6335 (FIG. 12, FIG. 13). These three second abutting portions 6333 to 6335 respectively have an arc shape extending along the circumferential direction centered around the center axis of the lens-barrel main body 631, and are respectively positioned rotationally symmetrically by 120′ about the center axis.

While FIG. 12 and FIG. 13 illustrate a case in which the second abutting portions 633 according to the second modification is applied to the illumination lens barrel 63 explained in the fifth embodiment described above, it may be applied to the illumination lens barrel 63 explained in the first to the fourth embodiments described above.

Also when the second abutting portions 633 according to the second modification explained above are adopted, an effect similar to those of the first to the fifth embodiments described above is produced.

The number of the second abutting portion 633 is not limited to one, two, or three, but four or more may be arranged.

### Third Modification

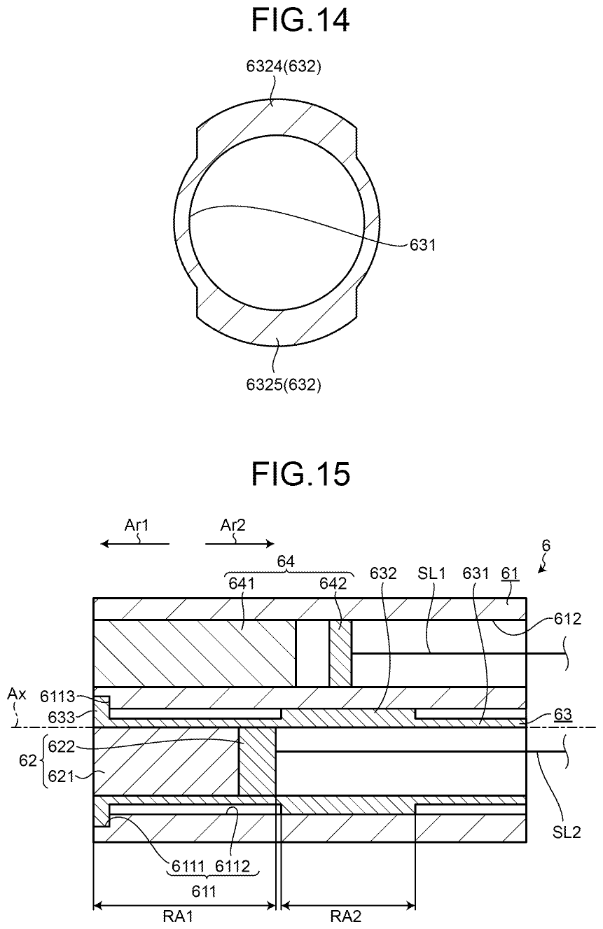

FIG. 14 is a diagram explaining a third modification of the first to the fifth embodiments. Specifically, FIG. 14 is a diagram corresponding to FIG. 3.

Although the first abutting portion 632 has a ring shape extending throughout the entire circumferential direction centered around the center axis of the lens-barrel main body 631 in the first to the fifth embodiments, it is not limited thereto, and a shape of the third modification illustrated in FIG. 14 may be adopted.

The first abutting portion 632 according to the third modification is arranged in two pieces. In the following, the two first abutting portions 632 are denoted as the first abutting portions 6324, 6325 (FIG. 14). These two first abutting portions 6324, 6325 respectively have an arc shape extending along the circumferential direction centered around the center axis of the lens-barrel main body 631, and are positioned rotationally symmetrically by 180′ about the center axis.

Also when the first abutting portion 632 according to the third modification explained above is adopted, an effect similar to those of the first to the fifth embodiments described above is produced.

The number of the first abutting portions 632 is not limited to one or two, but three or more may be arranged. Moreover, when the first abutting portion 632 is arranged in plurality, the arrangement position of the first abutting portions 632 may be shifted in a direction along the center axis Ax.

### Fourth Modification

FIG. 15 is a diagram explaining a fourth modification of the first to the fifth embodiments. Specifically, FIG. 15 is a diagram corresponding to FIG. 2.

Although the illumination unit 62 is constituted only of the illumination optical system 621 in the first to the fifth embodiments, not limited thereto, it may be constituted of the illumination optical system 621 and a light emitting device 622 as the fourth modification illustrated in FIG. 15.

The light emitting device 622 is electrically connected to the control device 5 by a signal line SL2, and emits light under control of the control device 5. The illumination optical system 621 irradiates the light from the light emitting device 622 to the inside of a living body from the distal end of the insertion portion 21. That is, in the fourth modification, the light guide LG and the light source device 4 are omitted.

While FIG. 15 illustrates a case in which the illumination unit 62 according to the fourth modification is arranged in the illumination lens barrel 63 explained in the first embodiment described above, it may be arranged in the illumination lens barrel 63 explained in the first to the fourth embodiments described above.

Also when the illumination unit 62 according to the fourth modification explained above, an effect similar to those of the first to the fifth embodiments is produced.

According to an imaging unit and an endoscope according to the disclosure, it is possible to suppress heat transfer from an illumination unit to an imaging-unit main body.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An imaging unit comprising:
an illumination unit that includes an illumination optical system;
a first holding frame configured to hold the illumination unit;
an imaging-unit main body that includes an imager, and an observation optical system configured to form the subject image on the imager; and
a second holding frame configured to hold the imaging-unit main body, wherein
a first abutting portion configured to abut on the second holding frame in a radial direction of the first holding frame is arranged on an outer surface of the first holding frame,
when a range holding the illumination unit in a direction along an optical axis of the illumination optical system in the first holding frame is a first axial-direction range, and a range in the direction along the optical axis different from the first axial-direction range is a second axial-direction range, the first abutting portion is arranged in the second axial-direction range, and
a cavity is formed by the first abutting portion in the first axial-direction range between the first holding frame and the second holding frame.

2. The imaging unit according to claim 1, wherein
the second axial-direction range is a range on a proximal end side relative to the first axial-direction range.

3. The imaging unit according to claim 1, wherein
when dividing the first axial-direction range evenly into two ranges of a range on a distal end side and a range on a proximal end side, the second axial-direction range is a range overlapping at least a part of the range on the proximal end side.

4. The imaging unit according to claim 1, wherein
the second axial-direction range is a range on a proximal end side relative to an arrangement position of the imager.

5. The imaging unit according to claim 1, wherein
the second axial-direction range is arranged in plurality in the direction along the optical axis.

6. The imaging unit according to claim 1, wherein
when dividing the first axial-direction range evenly into two ranges of a range on a distal end side and a range on a proximal end side, the second axial-direction range is a range overlapping at least a part of the range on the distal end side.

7. The imaging unit according to claim 1, wherein
the first abutting portion is configured to determine a position of the first holding frame in a radial direction by abutting on the second holding frame.

8. The imaging unit according to claim 1, wherein
on the outer surface of the first holding frame, a second abutting portion configured to abut on the second holding frame in the direction along the optical axis is arranged, the second abutting portion being positioned on a distal end side relative to the first abutting portion.

9. The imaging unit according to claim 8, wherein
the second abutting portion is configured to determine a position of the first holding frame in the direction along the optical axis by abutting on the second holding frame.

10. The imaging unit according to claim 8, wherein
the first abutting portion and the second abutting portion are integrally formed.

11. The imaging unit according to claim 1, wherein
the illumination optical system includes a lens constituted of two layers of a core portion and a cladding portion arranged on an outer circumference of the core portion, and
the second axial-direction range is a range overlapping at least a part of an arrangement position of the lens.

12. The imaging unit according to claim 1, wherein
the first abutting portion has a ring shape extending throughout an entire circumferential direction centered around a center axis of the first holding frame.

13. The imaging unit according to claim 1, wherein
the first abutting portion has an arc shape extending along a circumferential direction centered around a center axis of the first holding frame.

14. The imaging unit according to claim 8, wherein
the second abutting portion has an arc shape extending along a circumferential direction centered around a center axis of the first holding frame.

15. An endoscope comprising:
an insertion portion configured to be inserted into an inside of a subject body; and
an imaging unit that is arranged at a distal end of the insertion portion, wherein
the imaging unit includes
an illumination unit that includes an illumination optical system configured to irradiate light to a subject;
a first holding frame configured to hold the illumination unit;
an imaging-unit main body that includes an imager configured to capture a subject image, and an observation optical system configured to form the subject image on the imager; and
a second holding frame configured to hold the imaging-unit main body,
on an outer surface of the first holding frame, a first abutting portion configured to abut on the second holding frame in a radial direction of the first holding frame is arranged,
when a range holding the illumination unit in a direction along an optical axis of the illumination optical system in the first holding frame is a first axial-direction range, and a range in the direction along the optical axis different from the first axial-direction range is a second axial-direction range, the first abutting portion is arranged in the second axial-direction range, and
a cavity is formed by the first abutting portion in the first axial-direction range between the first holding frame and the second holding frame.
